(19) United States
(12) Patent Application Publication
(10) Pub. No.: US 2011/0015576 A1
(43) Pub. Date: Jan. 20, 2011

(54) MEDICAMENT IDENTIFICATION SYSTEM FOR MULTI-DOSE INJECTION DEVICES

(75) Inventors: David Aubrey PLUMPTRE, Droitwich Spa (GB); Christopher James SMITH, Holmes Chapel (GB)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 12/788,790

(22) Filed: May 27, 2010

Related U.S. Application Data
(60) Provisional application No. 61/182,848, filed on Jun. 1, 2009.

(30) Foreign Application Priority Data
Jul. 10, 2009 (EP) 09009043.2

(51) Int. Cl. A61M 5/31; B23P 17/04

(52) U.S. Cl. 604/189; 29/428

(57) ABSTRACT

A dynamic identification system for a multi-dose injection device includes a dose dial sleeve containing indicia of the medicament contained within the device becomes visible or available to the olfactory or gustation senses only during dose setting as the dial sleeve is translated proximally out of the outer housing of the device. A user can readily identify the medicament contained within the device as the dose is being set. A static identifier located on the device that matches the dynamic identifier on the dial sleeve can also be used as a medicament identifier.

### BACKGROUND

### 1. Field of the Present Patent Application

The present patent application is generally directed to drug delivery devices. More particularly, the present patent application is generally directed to drug delivery devices, such as pen type drug delivery devices. Such devices provide for self administration of medicinal product from a multi-dose cartridge and permit a user to set the delivery dose or set a single fixed dose. In particular, the present invention relates to a dynamic identification system for such injectors where the user can easily determine or distinguish the type of medication contained within the cartridge by visual observation and will receive a reinforcing confirmation of that visual identifier during the act of dose setting. The present application may find application in both resettable (i.e., reusable) and non-reusable (i.e., non-resettable) type drug delivery devices. However, aspects of the invention may be equally applicable in other scenarios as well.

### 2. Background

Pen type drug delivery devices have application where regular injection by persons without formal medical training occurs. This is increasingly common among patients having diabetes where self-treatment enables such patients to conduct effective management of their disease.

Pen-type injectors are well known and all universally use some form of cartridge capable of delivering multiple doses of a specific type of medicine, such as human growth hormone or insulin. For a number of end users of such devices (typically patients being prescribed medicines) several injectors are needed to dispense a number of different medicaments. For example, diabetic patients may need one injection device containing long lasting insulin and a second injector containing short acting insulin. Clearly, it is important for such patients to know with absolute certainty what medicine is contained within which injection device. This is especially true for elderly patients, particularly for those who are visually impaired. Although manufacturers of medicament cartridges typically use some form of identification (lettering, color and/or symbols) on the labels affixed to the cartridges, this form of identification is often subtle and not readily apparent to certain types of users of such devices. One manufacturer has previously used small plastic chips of various colors so a user can connect to a portion of the device as an identification of the medicament. Unfortunately, these chips are very small and not easily replaceable. Another manufacturer, as explained in U.S. Pat. No. 5,693,027, supplies a color-coded adaptor top to fit on the end of the cartridge to assist in distinguishing the medicament. Again, these adaptor tops are relatively small and not distinct enough to allow certain users to easily recognize the medicament contained in the device. Some manufacturers of certain types of disposable injectors will color various parts of the device, like the housing and cap, in an attempt to distinguish devices containing different medicaments, but again the variations in color are often subtle and not easily recognized by particular users. One problem with known identification systems is that they are static in nature and the user becomes accustomed to the indicia and thus ignores the significance of the indicia.

Accordingly, there still exists a strong need to provide users of such devices with a simple and clear means to determine and distinguish the type of medicine that is contained in the devices. Moreover, it is important to constantly reinforce to the user the indicia that identifies the medicament contained within the device otherwise the user will tend to ignore or “look through” static indicia. By providing a dynamic indicia through vision, tactile feel, olfactory or gustation the user will have a stronger association with that form of identification as it relates to a specific medicament.

My invention solves the above-described problems by providing a dynamic identification system to a multi-dose injection device where an indicia of the medicament contained in the device progressively appears every time a user dials a dose. This dynamic identifier can take the form of color, tactile, lettering, smell, taste, label or combination of any of those forms and is preferably located on a part of the device that “appears” each time a dose is set. This dynamic identification system can also be used with one or more a static identifiers, such as a label or colored band, to provide the user with a clear and simple visual and/or tactile form of identifying the particular medicament contained in the device. These and other advantages will become evident from the following more detailed description of the invention.

### SUMMARY

According to an exemplary arrangement, a dose setting mechanism for a drug delivery device is provided where the mechanism comprises a body and a dial sleeve. The dial sleeve is operably connected with the body and has an outer surface that contains an indicia to identify a medicament that is contained within the drug delivery device. The indicia is hidden from view when a zero dose is set and is at least partially exposed when a dose greater than zero is set.

In another embodiment a dose setting mechanism for a drug delivery device is provided where the mechanism comprises an outer body, an inner body having a helical groove along an external surface of the inner body, and a dial sleeve disposed between the outer body and the inner body. The dial sleeve contains an indicia of the medicament contained in the device. Because the dial sleeve is rotatably engaged with the helical groove of the inner body, when a dose is set the dial sleeve is rotated with respect to both the outer body and the inner body and the dial sleeve is translated away from the outer housing to dynamically reveal the indicia to the user of the device.

In one embodiment of my identification system, the outer surface of the dial sleeve can be pigmented with a color to allow a user of the injection device to visually identify and distinguish the device as containing a specific type of medicament. In another embodiment, the indicia can be letters that spell a particular trade name of the medicament. Likewise, the indicia can be raised letters or symbols such as geometric patterns or Braille characters.

Single or multiple colors or designs can be used on different devices to allow a user to associate a particular color (or visual design) with a specific medicament. For example, a green colored dial sleeve could designate short acting insulin and a yellow dial sleeve on another device would designate long acting insulin. For reusable injection devices with replaceable cartridges, it is preferred to have the color, text, or design on the dial sleeve match that which is used on the label affixed to the cartridge. Because the indicia on the dial sleeve does not appear until the user begins to set a dose, the indicia, as it progressively appears to the user during dose setting, acts as a constant reminder and reinforcement system that greatly increases the chance that the user will remember the association between the identifier and the specific medicament.

In yet other embodiments, the indicia can be in the form of a unique taste when a patient licks the dial sleeve. Alternatively, the dial sleeve can transmit a distinctive smell to allow a user to distinguish different injection devices. Of course, taste and smell can be combined on the dial sleeve as well.

These as well as other advantages of various aspects of the present invention will become apparent to those of ordinary skill in the art by reading the following detailed description, with appropriate reference to the accompanying drawings.

### BRIEF DESCRIPTION OF THE DRAWINGS

Exemplary embodiments are described herein with reference to the drawings, in which:

FIG. 1 illustrates an arrangement of the drug delivery device in accordance with the one aspect of the present invention;

FIG. 2 illustrates the drug delivery device of FIG. 1 with the protective cap removed to reveal the cartridge holder containing a cartridge medicament, where the dial sleeve is extended proximally from the housing in a dose setting condition revealing a color indicia on the dial sleeve;

FIG. 3 illustrates the drug delivery device of FIG. 1 with the protective cap removed to reveal the cartridge holder containing a cartridge medicament, where the dial sleeve is extended proximally from the housing in a dose setting condition revealing a text indicia on the dial sleeve; and

FIG. 4 illustrates the drug delivery device of FIG. 1 with the protective cap removed to reveal the cartridge holder containing a cartridge medicament, where the dial sleeve is extended proximally from the housing in a dose setting condition revealing a design and/or a tactile indicia on the dial sleeve.

### DETAILED DESCRIPTION

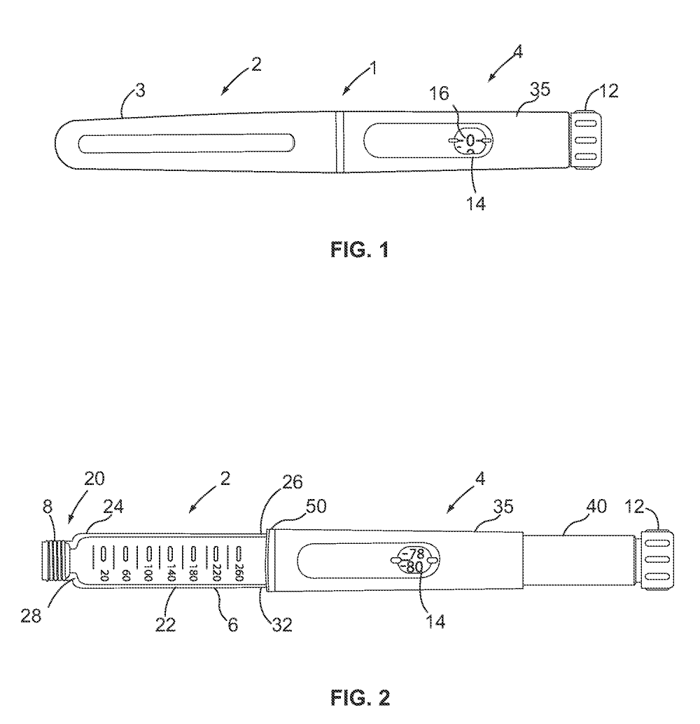

Referring to FIG. 1, there is shown a drug delivery device 1 in accordance with a first arrangement of the present invention. The drug delivery device 1 comprises a housing having a first cartridge retaining part 2, and dose setting mechanism 4. The drug delivery device may be a reusable drug delivery device or alternatively a disposable drug delivery device. By disposable device it is meant an injection device that is obtained from the manufacturer preloaded with medicament and cannot be reloaded with new medicament after the initial medicament is exhausted. The device may be a fixed dose or a settable dose, but in either case it is a multi-dose device. A first end of the cartridge retaining means 2 and a second end of the dose setting mechanism 4 are secured together by connecting features. For disposable devices, these connecting features would be permanent and for reusable devices, these connecting features would be releasable. The drug delivery device could also include syringes or other devices that have a dial sleeve, plunger, or other setting member that the user translates outwards, pulls or pushes, or cocks, including pre-filled single dose devices.

In this illustrated arrangement, the cartridge retaining means 2 is secured within the second end of the dose setting mechanism 4. A removable protective cap 3 is releasably retained over a second end or distal end of a cartridge retaining part or cartridge housing. The dose setting mechanism 4 comprises a dose dial grip 12 and a window or lens 14. A dose scale arrangement 16 is viewable through the window or lens 14. To set a dose of medication contained within the drug delivery device 1, a user rotates the dose dial grip 12, which in turn rotates dial sleeve 40 such that a dialed dose will become viewable in the window or lens 14 by way of the dose scale arrangement 16.

FIG. 2 illustrates the medical delivery device 1 of FIG. 1 with cover 3 removed from a distal end 20 of the medical delivery device 1. This exposes the cartridge housing 6. As illustrated, a cartridge 22 from which a number of doses of a medicinal product may be dispensed, is provided in the cartridge housing 6. Preferably, the cartridge 22 contains a type of medicament that must be administered relatively often, such as once or more times a day. One such medicament is either long acting or short acting insulin or an insulin analog. The cartridge 22 comprises a bung or stopper (not illustrated) that is retained near a second end or a proximal end 32 of the cartridge 22.

The cartridge housing 6 has a distal end 24 and a proximal end 26. Preferably, the cartridge distal end 24 of the cartridge housing 6 comprises a groove 8 for attaching a removable needle assembly however other needle assembly connection mechanisms could also be used. If the drug delivery device 1 comprises a resettable device, the cartridge proximal end 26 is removably connected to the dose setting mechanism 4. In one preferred embodiment, cartridge housing proximal end 26 is removably connected to the dose setting mechanism 4 via a bayonet connection. However, as those of ordinary skill in the art will recognize, other types of removable connection methods such as threads, partial threads, ramps and detents, snap locks, snap fits, and luer locks may also be used. The cartridge housing 6 further comprises an inner end face 28 near the first end or distal end 24 of the cartridge housing 6. Preferably, in order to maintain dose accuracy, the cartridge 22 is pressed up against or abuts this inner end face 28.

As previously mentioned, the dose setting mechanism 4 of the drug delivery device illustrated in FIG. 2 may be utilized as a reusable drug delivery device. (i.e., a drug delivery device that can be reset) Where the drug delivery device 1 comprises a reusable drug delivery device, the cartridge 22 is removable from the cartridge housing 6. The cartridge 22 may be removed from the device 1 without destroying the device 1 by merely having the user disconnect the dose setting mechanism 4 from the cartridge housing 6. Typically, each replaceable cartridge contains a label identifying the medication with a trade name, like Lantus® for insulin, or with a color or with a design or a combination of these three indicia forms. Unfortunately, once the cartridge is placed in the cartridge housing 6 the indicia may become difficult to see by certain users or is obscured by the dose scale that is typically printed on most cartridge housings.

In use, once cap 3 is removed, a user can attach a suitable needle assembly to the groove 8 provided at the distal end 24 of the cartridge housing 6. Such needle assembly may be screwed onto a distal end 24 of the housing 6 or alternatively may be snapped onto this distal end 24. After use, the replaceable cap 3 may be used to re-cover the cartridge housing 6. Preferably, the outer dimensions of replaceable cap 3 are similar or identical to the outer dimensions of dose setting mechanism 4 so as to provide an impression of a unitary whole as illustrated in FIG. 1 when replaceable cap 3 is in position covering cartridge housing 6 when the device is not in use.

FIG. 1 shows the device in a zero dose setting position as evidenced by the “0” showing through window 14. In the zero dose position dial sleeve 40 (see FIG. 2) is hidden because it does not extend in the proximal direction away from the outer housing 35. In other words, the only visible part of the dial sleeve is the numbering seen through the window 14. At this zero dose setting position the indicia on the dial sleeve is not visible to the user. Referring now to FIGS. 2-4, the user has set a dose of 79 units as indicated by the dose numbers seen through window 14. The dial sleeve 40 has moved or translated outwardly in the proximal direction away from the outer housing 35. To arrive at this position the user started from the zero dose position and began to rotate dose dial grip 12 causing dial sleeve 40 to also rotate and move axially in a proximal direction revealing or exposing more and more of the dial sleeve as the final dose of 79 units was reached. The dial sleeve can be manufactured as one or more parts that are assembled together such that all the parts move as a unitary part. For example, a distal end portion maybe made of white plastic with black dose numbers to provide maximum contrast. Likewise, different materials of construction may be used for each portion for cost or wear and tear considerations. Manufacturing the dial sleeve in separate sections may also make it easier to add the dynamic indicia to the most proximal section of the dial sleeve.

As illustrated in FIG. 2 the outer surface of the dial sleeve 40 can be pigmented or painted a unique color that preferably matches or corresponds with the color used to designate the medicament contained within cartridge housing 6. The dial sleeve can be pigmented with a color or colors that is associated with a particular medicament and that the user can easily visualize. The particular method or color used to pigment the band is not critical to my invention; however, bright colors are particularly preferred for users with poor or limited vision provided of course that there is some connection of the color chosen to specific medicaments available for use in the device.

As the user is setting the desired dose more and more of the color of the dial sleeve dynamically appears and is readily noticed by the user. This progression of uncovering more of the indicia as the dose is being set reinforces and reminds the user about the medicament that is present in the device. In an alternative embodiment a static indicia of the medicament may be used in conjunction with the dynamic indicia. For example, as shown in FIG. 2, the device manufacturer might incorporate band 50 that matches the color on the dial sleeve on housing 35 as a non-removable static indicia. Alternatively, the static indicia may be removable and added by a medical practitioner or by the user.

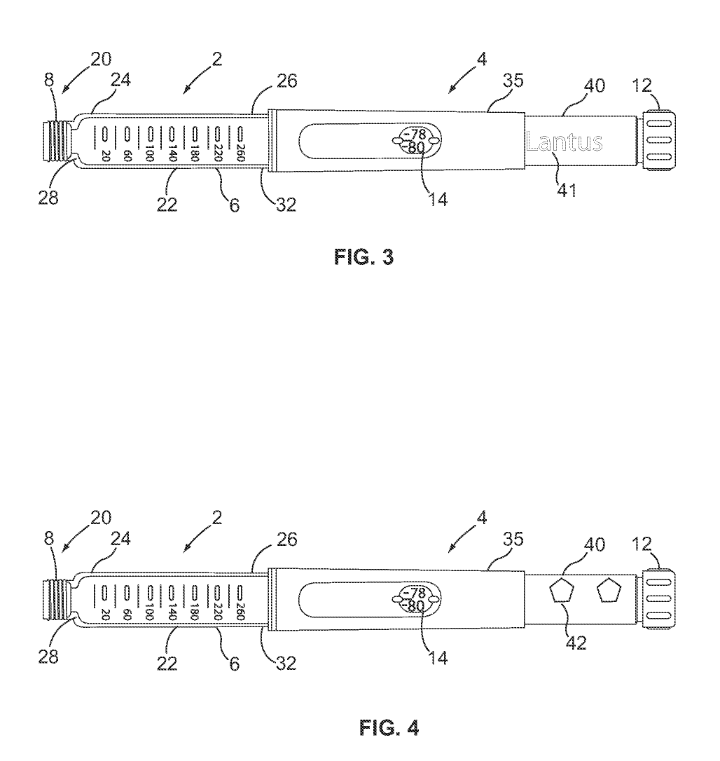

In those circumstances where the user might be color blind, then text or symbols can be used on dial sleeve 40 in conjunction with or without color. FIG. 3 shows one such possible configuration where the trade name of the medicament (“Lantus”) is printed (or applied using a label) on the colored dial sleeve. As the user begins to set a dose the letters begin to appear, i.e. first “s,” then “u,” then “t,” and so on depending on the amount of dose to be set. This dynamic progression of reveled letters works to alert the user to the type of medicament contained in the device.

Alternatively, in those circumstances where the user's eyesight is impaired or non-existent, then the outer surface of dial sleeve 40 can be textured to tactilely identify and distinguish the device as containing a specific type of medicament. This texturing may take any form, such as a raised design or even lettering, like Braille, provided that the user can easily recognize it. An example of such texturing is shown in FIG. 4 where raised pentagon symbols 42 are located on the outer surface of dial sleeve 40. These symbols preferably would match the symbols used to identify a specific medicament and would preferably appear on the label of the medicament. Additionally, a scent, a flavor, or both could be added to the dial sleeve to allow a visually impaired patient to associate a unique taste and/or smell of the device to a specific medicine. Likewise, both coloring and texturing could be also be used on the dial sleeve. In summary, the indicia can be selected from the group consisting of text, numbers, labels, tags, geometric designs, Braille figures, colors, tactile shapes, flavors, smells and combinations of these.

Exemplary embodiments of the present invention have been described. Those skilled in the art will understand, however, that changes and modifications may be made to these embodiments without departing from the true scope and spirit of the present invention, which is defined by the claims.

1. A dose setting mechanism for a drug delivery device, the mechanism comprising:
an outer body;
an inner body having a helical groove along an external surface of the inner body; and
a dial sleeve that is disposed between the outer body and the inner body, where the dial sleeve has an inner surface that is,
rotatably engaged with the helical groove of the inner body; and
contains an indicia on the outer surface to identify a medicament in the drug delivery device, where the indicia is hidden from view when a zero dose is set, wherein when a dose is set, the dial sleeve is rotated with respect to both the outer body and the inner body causing the dial sleeve to translate away from the outer housing exposing the indicia on the outer surface of the dial sleeve.

2. The dose setting mechanism of claim 1 wherein the indicia is selected from the group consisting of text, numbers, labels, tags, geometric designs, Braille figures, colors, tactile shapes and combinations of these.

3. The dose setting mechanism of claim 1 wherein the outer surface of the dial sleeve is pigmented to allow a user of the drug delivery device to visually identify and distinguish the device as containing a specific type of medicament during setting of a dose.

4. The dose setting mechanism of claim 1 wherein said dial sleeve has a generally smooth outer surface and contains printed letters, numbers, color, geometric designs or a combination of these indicia forms.

5. The dose setting mechanism of claim 1 wherein the outer surface of the dial sleeve is textured to allow a user of the drug delivery device to tactilely identify and distinguish the device as containing a specific type of medicament.

6. The dose setting mechanism of claim 1 where the dial sleeve comprises two or more sections that are connected together during assembly so that the connected sections move together in unison.

7. The dose setting mechanism of claim 6 where a first section of the dial sleeve provides a surface onto which the dose numbers are printed and a second section is pigmented to provide the required indication of drug type.

8. A method of providing a dynamic indicia of medication to a drug delivery device comprising adding a scent, flavor or a combination of scent and flavor to a dose setting member or dose delivery member of an injection device accessible to the user before dose delivery.

9. A dose setting mechanism for a drug delivery device, the mechanism comprising:
a body; and
a dial sleeve that is operably connected with the body, where the dial sleeve has an outer surface that contains an indicia to identify a medicament and where the indicia is hidden from view when a zero dose is set;
wherein at least part of the indicia on the outer surface of the dial sleeve is exposed when a dose greater than zero is set.
